(19) United States
(12) Patent Application Publication
(10) Pub. No.: US 2009/0196959 A1
(43) Pub. Date: Aug. 6, 2009

(54) Fresh Pineapple Spear Pasteurization Process and Product

(75) Inventor: Miguel Guzman, Aptos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fresh Express, Inc., Salinas, CA (US)

(21) Appl. No.: 12/027,237

(22) Filed: Feb. 6, 2008

(51) Int. Cl. B65B 25/04; B65B 55/14; A23B 7/10; A23B 7/02; G01N 33/02

(52) U.S. Cl. 426/106; 426/392; 426/232

(57) ABSTRACT

A process for producing a fresh pineapple spear package product from fresh whole pineapple is disclosed. The process includes removing the crowns from the fresh whole pineapples; removing surface soil and microbial loads from the fresh whole pineapples; eliminating the rind from the fresh whole pineapples to form peeled fresh pineapples; eliminating the core from the fresh whole pineapples to form cored and peeled fresh pineapples; forming fresh pineapple spears from the cored and peeled fresh pineapples; packaging the fresh pineapple spears in sealed heat-resistant packages to form packaged fresh pineapple spears; and pasteurizing the fresh pineapple spears in the sealed heat-resistant packages to form the fresh pineapple spear package product.

### BACKGROUND OF THE INVENTION

The present invention relates to a method of treating fresh pineapples to prepare them for marketing so that the fresh pineapple product is preserved for a longer period of time in a form free from spoilage, and yet maintains its desirable fresh texture and appearance.

There is a growing desire to eat healthy snacks. Current dietary guidelines recommend the consumption of two cups of fruit per day at the 2000-calorie reference diet. An example of two cups of fruit includes: one small banana, one large orange, and one-fourth cup of dried apricots or peaches. Some guidelines suggest that eating more than five portions of fruit and vegetables a day can substantially reduce the risk of the stroke. However, most people do not eat the recommended fruit portions for many reasons. One reason is the inconvenience of having to clean, peel, and cut the fresh fruit before it can be consumed. At least for this reason many prefer the convenience of packaged goods or even fruit juices, which only require one to open the package in order to eat its contents. However, it is known that the whole fruit, as opposed to fruit juices, has more fiber, and it is more filling.

Some fruit, such as pineapples, are not readily consumable in their whole fruit form. In addition, the cleaning, peeling and cutting that is required for pineapples, discourages most consumers from purchasing and eating pineapples. As such, packaged or canned pineapples are sometimes consumed more than fresh pineapples. However, packaged fruit, while being more convenient, may not be as appealing or even as nutritious as fresh fruit.

A wide variety of fresh fruit preservation methods are used in the food industry. Most of those methods are based on drying the fruit, which results in a product with a reduced appeal to the average consumer, as well as a reduced nutritional value, since the relative content of the carbohydrates is increased in the dried fruit. Other methods add fruit juices with a specific brix/acid ratio to the fresh fruit. This increases the shelf life of the fruit, but also increases the cost and reduces the appeal of thus preserved fruit. Some other methods package fresh cut fruit, but a vacuum packaging is required to achieve the reduced microbial content. The vacuum packaging increases the shelf life of the fruit, but results in a higher packaging cost and a reduced appeal to an average consumer. Some methods for fresh fruit preservation use two pasteurization steps: before the packaging and after the packaging. The two pasteurization steps add additional cost to the process, and may affect the taste of the fruit.

There is therefore a need for a fresh pineapple fruit product that is conveniently packaged for consumption and one that is preservable for long periods of time in a form free from spoilage and which maintains the fruit's desirable fresh texture and appearance.

### BRIEF SUMMARY OF THE INVENTION

The present invention is directed toward a process for producing a fresh pineapple spear package product from fresh whole pineapples. In one embodiment, the process includes: removing the crowns from the fresh whole pineapples; removing surface soil and microbial loads from the fresh whole pineapples; eliminating the rind from the fresh whole pineapples to form peeled fresh pineapples; eliminating the core from the fresh whole pineapples to form cored and peeled fresh pineapples; forming fresh pineapple spears from the cored and peeled fresh pineapples; packaging the fresh pineapple spears in sealed heat-resistant packages to form packaged fresh pineapple spears; and pasteurizing the fresh pineapple spears in the sealed heat-resistant packages to form the fresh pineapple spear package product.

In one aspect, removing surface soil and microbial loads from the fresh whole pineapples is done using direct steam, or hot water, or chemical surface disinfectants.

In another aspect, pasteurizing the fresh pineapple spears in the sealed packages is done in a hot water bath for approximately 5-15 minutes.

In another aspect, pasteurized sealed packages are chilled by exposing the packaged and pasteurized fresh pineapple spears to a cold water bath at temperature of approximately 34° F. until the product temperature falls below 40° F.

In yet another aspect, the sealing of the fruit in the heat resistant packages is done without forming vacuum packaging.

For a further understanding of the nature and advantages of the invention, reference should be made to the following description taken in conjunction with the accompanying figures. It is to be expressly understood, however, that each of the figures are provided for the purpose of illustration and description only and is not intended as a definition of the limits of the embodiments of the present invention.

### BRIEF DESCRIPTION OF THE DRAWINGS

FIG. 1 is a simplified flowchart of the process for producing a pasteurized pineapple spear product in accordance with one embodiment of the present invention. FIG. 1 describes the process steps from the receiving and storage of the raw fruit to the storage of the shipment-ready packages.

### DETAILED DESCRIPTION OF THE INVENTION

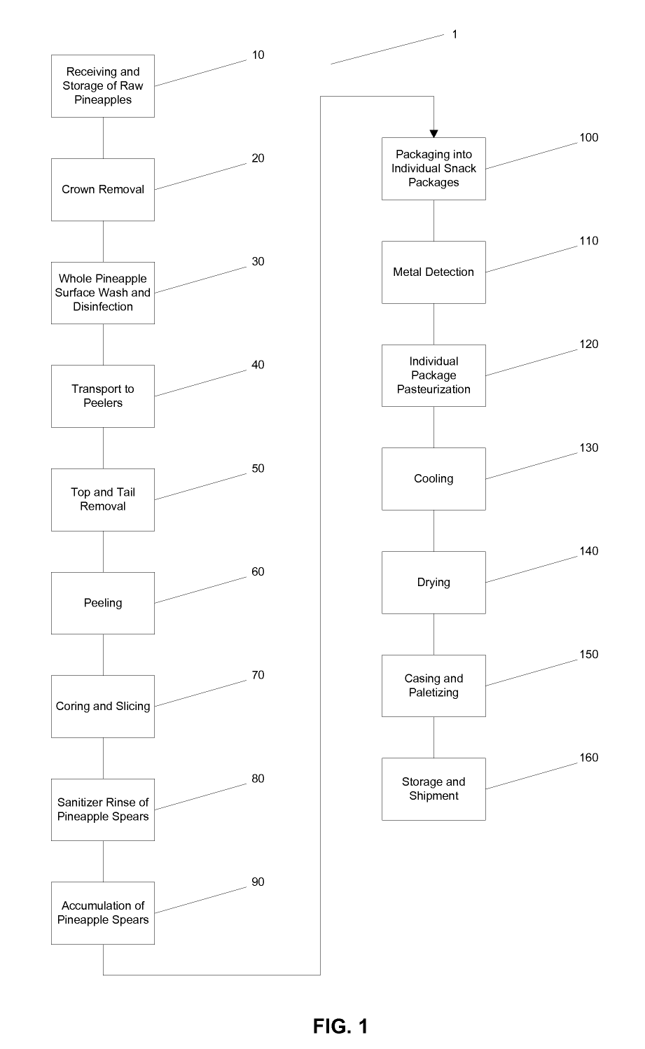

A method for preparing packaged fresh fruit with increased shelf life under unfavorable temperature condition is described below. The invention is described in detail with reference to the process steps illustrated in FIG. 1.

As shown in step 10 of FIG. 1, the incoming pineapple fruit is received and stored. The incoming quality parameters, such as variety, defects, age, origin, Brix, temperature, etc. can be verified in this step. The incoming fruit can be segregated according to different types of fruit products, while the substandard fruit is eliminated from the manufacturing process. This incoming inspection is typically done using equipment such as a refractometer or a thermometer, either in a batch or a continuous processing mode.

At step 20 the pineapple crown is removed by hand. This step is typically, but not necessarily, the first process step in removing the inedible pineapple parts. Pineapple crown removal can also be done by a variety of mechanized ways.

At step 30 the pineapple surface is washed and disinfected. Typically, high pressure water showers and steam pasteurizers can be used to remove surface soil and microbial contamination. This step may consist of one or multiple passes through the showers and pasteurizers. The pressure and temperature of water or steam, as well as the duration of the fruit exposure, can be adjusted to achieve the desired outcome with the smallest expenditure of time and material, i.e. water or steam. In addition or instead of water showers and steam pasteurizers, chemical surface disinfection of the fruit surface can be used. Commonly used chemical sanitizers in the food industry are peracetic acid, chlorine, ozone, and chlorine dioxide. Other chemical sanitizers may also be used.

Next, at step 40 the pineapples are transported to peelers. This can be done in either a continuous or batch mode, using, for instance, moving belt conveyors or carts. The means of transportation should be gentle, without too much vibration or shock that could damage the pineapples.

At step 50 the top and tail of the pineapple fruit are removed. This can be done either by hand or by one of the many mechanized process, which are well known to the person skilled in the art. The rind created in the process is removed off the work surface in this step.

At step 60 the pineapple fruit is peeled. The rind is peeled off the edible core using cylindrically shaped peelers, either hand operated or mechanized. As a result, a pineapple cylinder is created. Preferably, at the end of this step there is very little rind left on the pineapple.

At step 70 the pineapple fruit is cored and sliced. The pineapple core is eliminated using a corer tool, which will create a fruit shaped as a hollow cylinder. The fruit is then sliced into spears using cutter tools. For example, the hollow cylinder fruit could be sliced into rings or cut into eight or more spears. Other numbers of fruit spears can be produced from the hollow cylinder using different cutting tools.

At step 80 the pineapple spears undergo a sanitizer rinse. The purpose of this step is to reduce micro organism count. An open mesh conveyor that transports the fruit beneath a sanitizer shower of a sanitizer (e.g. Tsunami-100 sanitizer) can be used in this embodiment, but similar sanitizing methods are also possible. Some of the commonly used sanitizers in the food processing industry are peracetic acid, chlorine, ozone, and chlorine dioxide, but many others are also known to a person skilled in the art. Ideally, all the surfaces of the fruit get in contact with the sanitizer to reduce micro organism count. Further micro organism count reduction is explained below at step 120.

At step 90 the sanitized pineapple spears are placed inside temporary storage boxes, where the spears are accumulated prior to being placed in the individual snack packages. The storage boxes can be made of perforated plastic totes, but other types of the temporary storage boxes may also be used.

At step 100 the individual snack packages are created by placing the pineapple spears in the sealed bags. The packages can be sealed with or without vacuuming them. The packages can resist the target pasteurization temperatures explained in step 120 below. The end consumers can purchase snacks in these same packages.

At step 110 the individual fruit packages are subjected to metal detection. In one embodiment, the packages are moving on the conveyor system with the metal detection system placed above the conveyor. At this step those snack packages which may have been contaminated with metal scraps in any of the upstream process steps are identified by the metal detector. The metal contaminated packages are subsequently rejected off the conveyor system. It will be appreciated that the step illustrated at step 110 can occur at any place in the process after the individual snacks are packaged, and before the packages leave the processing facility for the retail delivery.

At step 120 the individual packages are pasteurized. Unlike in the microbial count reduction at step 80, the microbial count reduction and enzyme inactivation at step 120 is done over the fruit that is sealed in the individual packages, which, as explained at step 100 above, can resist pasteurization temperatures. In one embodiment, continuous steam blanchers producing steam at about 180-212° F. are used for pasteurization by subjecting the packages to the steam for about 5 to 15 minutes. A variety of temperature vs. time combinations can be used to achieve the desired goals of reducing the microbial count and inactivating pineapple enzymes. For instance, hot water or another high temperature medium can be used instead of the water steam.

At step 130 the finished product is cooled. In one exemplary embodiment, a continuous hydro-chiller with chilled water at 34° F. can be used to cool the packages below 40° F. At this step, the temperature of the finished product is reduced quickly, thereby limiting the product's exposure to temperature ranges that are conducive to rapid micro organism count growth.

At step 140 the individual fruit packages are dried off. The moisture that was left on the packages after the cooling step 130 can now be removed by, for instance, a sonic air knife with an indexing conveyor. Other moisture removing methods can be used for drying the individual fruit packages, like manually wiping the packages or blowing dry air over the packages.

At step 150 the individual pineapple spear packages are placed in cases and palletized. Other means for packaging the individual pineapple spear packages in a shipment worthy packaging may also be used.

Next, at step 160 the individual pineapple spear packages are stored prior to sale to the end consumer.

Normally, optimum storage temperature for pineapple spears is below 33° F. if the fruit is to be stored for multiple days, but, because of the above described pasteurization process, the pineapple spears in the individual packages can withstand temperatures in the range of 33° F. to 45° F. for up to 30 days. The present invention is especially well suited for the, so called, abusive refrigerated storage temperatures whereby the storage temperature can stay between 40 to 45° F. for the excessively long period of time, like, for example, 21-30 days.

The present invention can slow down the pineapple deterioration as well as gas formation by the microorganisms, which would otherwise create an inflated package that is less desirable in the eyes of the consumers. Another advantage of the present invention is that the fresh fruit packages produced by the present invention have a moisture content that is not substantially altered from the moisture content of the fresh whole pineapples. Typically, the process described above results in the moisture content of the fruit reduced by up to about 15%, producing the fruit with the moisture content of around 85% or higher in the outgoing product. This is not the case with many other fruit preservation processes which are targeted at storage temperature above 33° F., because those processes are based on the dried fruit with a significantly lower final moisture content than the one in the present invention.

The present invention can be applied to fruits other than pineapple by using appropriate tooling and process changes. For instance, apples, pears, or plums could be processed in a manner similar to the one described above. A wide variety of the fruit processing tools exists on the market, and a person skilled in the art would know how to choose suitably sized and shaped tools for a particular fruit type. The number and size of fresh fruit spears could also be adjusted based on the type of the incoming fresh fruit. Sanitizers that are well suited for a particular type of fruit as well as the appropriate adjustments for the pasteurization temperature and time duration could be used. When applied to other fruits, the process in accordance with the principle of the invention would result in a packaged fruit having similar advantages over the fruit preserving methods that produce the dried fruit.

The above description is illustrative, and is not restrictive. Many variations in the equipment and manufacturing process will become apparent to those skilled in the art upon review of the disclosure. The scope of the invention should, therefore, be determined not with reference to the above description, but instead should be determined with reference to the pending claims along with their full scope or equivalents. And, as will be understood by those skilled in the art, the present invention may be embodied in other specific forms without departing from the essential characteristics thereof. These other embodiments are intended to be included within the scope of the present invention, which is set forth in the following claims.

What is claimed is:

1. A process for producing a fresh pineapple spear package product from fresh whole pineapples, comprising:
removing the crowns from the fresh whole pineapples;
removing surface soil and microbial loads from the fresh whole pineapples;
eliminating the rind from the fresh whole pineapples, to form peeled fresh pineapples;
eliminating the core from the fresh whole pineapples, to form cored and peeled fresh pineapples;
forming fresh pineapple spears from the cored and peeled fresh pineapples;
packaging the fresh pineapple spears in sealed heat-resistant packages to form packaged fresh pineapple spears; and
pasteurizing the fresh pineapple spears in the sealed heat-resistant packages to form the fresh pineapple spear package product.

2. The process of claim 1, wherein said removing surface soil and microbial loads from the fresh whole pineapples comprises direct steam, or hot water, or chemical surface disinfection of the fresh whole pineapples.

3. The process of claim 1, further comprising rinsing the fresh pineapple spears with a food processing sanitizer.

4. The process of claim 3, wherein said food processing sanitizer is chosen from a group consisting of peracetic acid, chlorine, ozone, chlorine dioxide, and combination thereof.

5. The process of claim 1, wherein said pasteurizing comprises submerging the packaged fresh pineapple spears in a hot water bath for approximately 5-15 minutes.

6. The process of claim 5, wherein said pasteurizing comprises submerging the packaged fresh pineapple spears in a hot water bath for approximately 5-7 minutes.

7. The process of claim 5, wherein said pasteurizing comprises submerging the packaged fresh pineapple spears in a hot water bath at approximately 180-212° F.

8. The process of claim 1, wherein said pasteurizing comprises exposing the packaged fresh pineapple spears to a continuous steam blancher for approximately 5-15 minutes.

9. The process of claim 8, wherein said pasteurizing comprises submerging the packaged fresh pineapple spears in a hot water bath for approximately 5-7 minutes.

10. The process of claim 8, wherein said pasteurizing comprises submerging the packaged fresh pineapple spears in a hot water at approximately 180-212° F.

11. The process of claim 1, further comprising cooling the packaged and pasteurized fresh pineapple spears.

12. The process of claim 11, wherein said cooling comprises exposing the packaged and pasteurized fresh pineapple spears to a cold water bath at a temperature of approximately 34° F.

13. The process of claim 12, wherein said cooling is performed until the product temperature falls below 40° F.

14. The process of claim 1, wherein said eliminating the rind from the fresh whole pineapples comprises topping, tailing and peeling the fresh whole pineapples.

15. The process of claim 1, wherein said packaging the fresh pineapple spears in a sealed heat-resistant package comprises sealing the packages without forming a vacuum packaging.

16. The process of claim 1, wherein said forming of fresh pineapple spears comprises forming a plurality of spears from each cored and peeled fresh pineapple.

17. The process of claim 1, further comprising detecting metal contaminants in the packaged fresh pineapple spears.

18. The process of claim 17, further comprising rejecting metal contaminated packaged fresh pineapple spears.

19. The process of claim 1, further comprising drying the fresh pineapple spear package, so as to minimize surface moisture on the fresh pineapple spear package.

20. The process of claim 19, wherein said drying is performed by an air knife conveyor system.

21. The process of claim 1, wherein the moisture content of the fresh pineapple spear product is not substantially altered from the moisture content of the fresh whole pineapples.

22. The process of claim 1, wherein the moisture content is approximately 85% by weight.

23. The process of claim 1, wherein the fresh pineapple spear product is a fruit product that is stable at refrigerated storage temperatures of approximately 40 to 45° F. for a period between approximately 21 to 30 days.

24. The process of claim 1, wherein the fresh pineapple spear product slows down the pineapple deterioration and gas formation by microorganisms that could create an inflated package.

25. The process of claim 1, wherein the nutritional value of the fresh pineapple spear product is not substantially altered when the packaged product is refrigerated at approximately 40 to 45° F. for a period between approximately 21 to 30 days.

26. A fresh pineapple spear package product made in accordance with a process comprising of:
removing the crowns from the fresh whole pineapples;
removing surface soil and microbial loads from the fresh whole pineapples;
eliminating the rind from the fresh whole pineapples, to form peeled fresh pineapples;
eliminating the core from the fresh whole pineapples, to form cored and peeled fresh pineapples;
forming fresh pineapple spears from the cored and peeled fresh pineapples;
packaging the fresh pineapple spears in sealed heat-resistant packages to form packaged fresh pineapple spears; and
pasteurizing the fresh pineapple spears in the sealed heat-resistant packages to form the fresh pineapple spear package product.

27. The product of claim 26, further comprising rinsing fresh pineapple spears with food processing sanitizer, wherein said food processing sanitizer is chosen from a group consisting of peracetic acid, chlorine, ozone, and chlorine dioxide, and combination thereof.
